(19) United States
(12) Patent Application Publication
(10) Pub. No.: US 2013/0109068 A1
(43) Pub. Date: May 2, 2013

(54) Methods For Producing Acetone, Butanol, and Ethanol

(71) Applicant: Utah State University, North Logan, UT (US)

(72) Inventors: Joshua T. Ellis, Logan, UT (US); Charles Miller, North Logan, UT (US); Ronald Sims, Logan, UT (US); Ashik Sathish, Logan, UT (US)

(73) Assignee: Utah State University, North Logan, UT (US)

(21) Appl. No.: 13/663,002

(22) Filed: Oct. 29, 2012

Related U.S. Application Data
(60) Provisional application No. 61/552,317, filed on Oct. 27, 2011.

(51) Int. Cl. C12P 7/28; C12P 7/06; C12P 7/16

(52) U.S. Cl. CPC C12P 7/28; C12P 7/16; C12P 7/065
USPC 435/150; 435/160; 435/161

(57) ABSTRACT

Methods of producing solvents from algae, where the methods include processing algae to yield processed biomass, fermenting the processed biomass with a Clostridium bacteria to yield solvents.

### CROSS-REFERENCE TO RELATED APPLICATIONS

This application claims priority to U.S. Provisional Patent Application No. 61/552,317, filed Oct. 27, 2011, the entirety of which is herein incorporated by reference.

### GOVERNMENT SPONSORED RESEARCH

This invention was made, at least in part, with government support under contract DE-EE0003114 awarded by the United States Department of Energy. The government has certain rights in the invention.

### TECHNICAL FIELD

The present disclosure relates to methods for producing solvents, more particularly, it relates to methods of making acetone, butanol, and ethanol (ABE) from algal biomass.

### BACKGROUND

Many clostridia species such as Clostridium beijerinckii, Clostridium saccharoperbutylacetonium, and C. acetobutylicum are anaerobic, saccharolytic, spore forming, and ABE producing bacteria that have been previously isolated from a variety of environments. Saccharolytic clostridia have been isolated from, for example, soils, lake sediments, well water, human feces, and canine feces. Clostridia are gram-positive, rod-shaped, motile (via flagella), and are obligate anaerobes.

ABE fermentation using C. acetobutylicum, as well as other ABE fermenting clostridia, along with various biological feedstocks have been investigated in the past. However, to meet the needs of renewable biofuels, improved methods for producing ABE from inexpensive, renewable feedstocks are needed.

### SUMMARY

The polysaccharides in algae biomass offers a tremendous amount of energy that can be harvested in the form of biofuels. The fermentation of carbohydrates to C2, C3, and C4 compounds such as ethanol, acetone, isopropanol, and butanol are definite in certain saccharolytic Clostridium spp., such as Clostridium acetobutylicum, Clostridium saccharoperbutylacetonium, and Clostridium beijerinckii. Acetone-butanol-ethanol (ABE) fermentation, utilizing algae as substrate, could be employed at industrial scales for the production of these high value solvents. Algal biomass would serve as an advantageous substrate due to its ubiquitous nature, as well as its advantages in application and bioconversion. Algae are considered to be the most important substrate for future production of clean and renewable energy. See, e.g., Demirbas, A., Use of algae as biofuel sources, Energy Conversion and Management, 51:2738-2749 (2010) Elsevier Ltd. Additionally, the use of algal biomass from wastewater treatment facilities would provide a cheap and renewable substrate, that could be continuously harvested, for the production of ABE.

The present disclosure in aspects and embodiments addresses these various needs and problems by providing methods of producing solvents from algae, the method comprising processing algae to yield processed biomass, fermenting the processed biomass with a Clostridium bacterium to yield solvents.

### BRIEF DESCRIPTION OF THE DRAWINGS

FIG. 1 illustrates a flow diagram according to an exemplary embodiment.

FIG. 2 illustrates production yields according to an exemplary embodiment.

FIG. 3 illustrates production yields according to an exemplary embodiment.

FIG. 4 illustrates production yields according to an exemplary embodiment.

FIG. 5 illustrates production yields according to an exemplary embodiment.

FIG. 6 illustrates production yields according to an exemplary embodiment.

FIG. 7 illustrates production yields according to an exemplary embodiment.

FIG. 8 illustrates production yields according to an exemplary embodiment.

### DETAILED DESCRIPTION

The present disclosure covers apparatuses and associated methods for producing ABE from algal biomass. In the following description, numerous specific details are provided for a thorough understanding of specific preferred embodiments. However, those skilled in the art will recognize that embodiments can be practiced without one or more of the specific details, or with other methods, components, materials, etc. In some cases, well-known structures, materials, or operations are not shown or described in detail in order to avoid obscuring aspects of the preferred embodiments. Furthermore, the described features, structures, or characteristics may be combined in any suitable manner in a variety of alternative embodiments. Thus, the following more detailed description of the embodiments of the present invention, as illustrated in some aspects in the drawings, is not intended to limit the scope of the invention, but is merely representative of the various embodiments of the invention.

In this specification and the claims that follow, singular forms such as “a,” “an,” and “the” include plural forms unless the content clearly dictates otherwise. All ranges disclosed herein include, unless specifically indicated, all endpoints and intermediate values. In addition, “optional” or “optionally” refer, for example, to instances in which subsequently described circumstance may or may not occur, and include instances in which the circumstance occurs and instances in which the circumstance does not occur. The terms “one or more” and “at least one” refer, for example, to instances in which one of the subsequently described circumstances occurs, and to instances in which more than one of the subsequently described circumstances occurs.

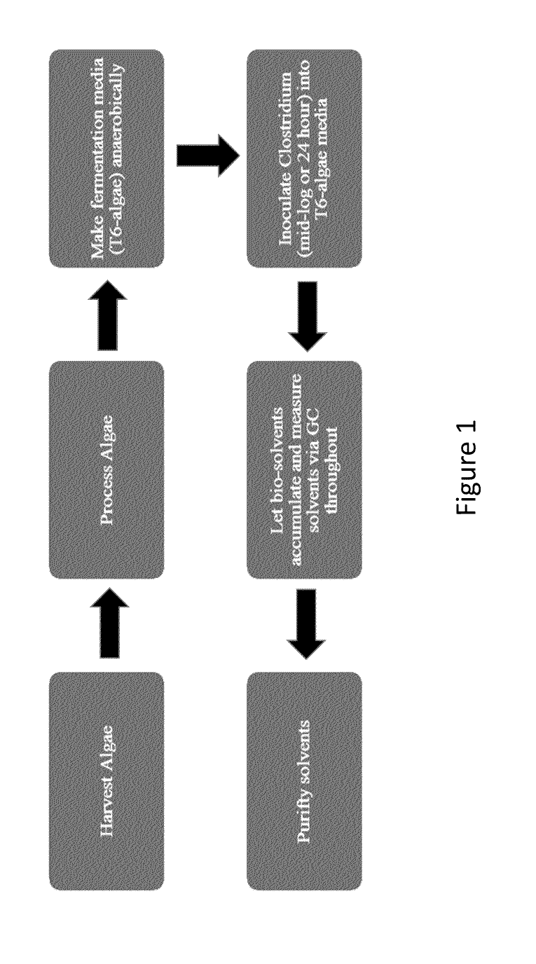

The present disclosure covers methods, compositions, reagents, and kits for making acetone, butanol, and ethanol (ABE) from algal biomass. A flow diagram of at least one embodiment is illustrated in FIG. 1.

I. Feed Stocks

As a feedstock, any suitable algae, cyanobacteria, or combination thereof may be used. In the description herein the terms “algae” or “algal” include algae, cyanobacteria, or combinations thereof. In embodiments, algae that produce high concentrations of polysaccharides may be preferred. In many embodiments, algae produced in waste water may be used. The algae may be lyophilized, dried, in a slurry, or in a paste (with for example 10-15% solid content).

After identification of a feedstock source or sources, the algae may be formed into a slurry, for example, by adding water, adding dried or lyophilized algae, or by partially drying, so that it has a solid content of from about 1-40%, such as about 4-25%, about 5-15%, about 7-12%, or about 10%.

The various steps to the process, according to some embodiments, are described in more detail below. The methods described herein may be accomplished in batch processes or continuous processes.

II. Algal Biomass Processing

The feedstock may be directly processed according to the ABE production described below. Alternatively, in some embodiments, the feedstock may optionally be processed into a processed biomass prior to ABE production as set forth in U.S. application Ser. No. 13/660,161, filed on Oct. 25, 2012, the entirety of which is herein incorporated by reference in its entirety. This processing to yield processed biomass may include cell lysis, and solid/liquid separation as described, for example, below.

A. Algal Cell Lysis

The algal cells may be optionally lysed by any suitable method, including, but not limited to acid and/or base hydrolysis (described below). Other methods may include mechanical lysing, such as smashing, shearing, crushing, and grinding; sonication, freezing and thawing, heating, the addition of enzymes or chemically lysing agents.

In some embodiments, the algal cells may be lysed by acid hydrolysis followed by an optional base hydrolysis.

(1) Acid Hydrolysis

To degrade the algal cells (or other cells present), to bring cellular components into solution, and to break down complex components, such as polysaccharides to their respective monosaccharide components as well as lipids to free fatty acids, a slurry of water and algae described above may be optionally heated and hydrolyzed with at least one acidic hydrolyzing agent. Complex carbohydrates may include, but are not limited to, starch, cellulose, and xylan. The degradation of these complex polysaccharides from the acid hydrolysis will yield oligosaccharides or monosaccharides that can be readily used for ABE production. These complex lipids may include, for example, triacylglycerols (TAGs), glycolipids, etc. In addition to degrading algal cells and complex lipids, the acidic environment created by addition of the hydrolyzing agent removes the magnesium from the chlorophyll molecules.

When heated, the slurry may reach temperatures of from about 1-200° C., such as about 20-100° C., about 50-95° C., or about 90° C. When temperatures above 100° C., or the boiling point of the solution are used, an apparatus capable of withstanding pressures above atmospheric pressure may be employed. In some embodiments, depending on the type of algae, the type and concentration of acid used for hydrolysis, the outside temperature conditions, the permissible reaction time, and the conditions of the slurry, heating may be omitted. Heating may occur prior to, during, or after addition of a hydrolyzing agent.

In addition, the slurry may be optionally mixed either continuously or intermittently. Alternatively, a hydrolysis reaction vessel may be configured to mix the slurry by convection as the mixture is heated.

Acid hydrolysis may be permitted to take place for a suitable period of time depending on the temperature of the slurry and the concentration of the hydrolyzing agent. For example, the reaction may take place for up to 72 hours, such as from about 12-24 hours. If the slurry is heated, then hydrolysis may occur at a faster rate, such as from about 15-120 minutes, 30-90 minutes, or about 30 minutes.

Hydrolysis of the algal cells may be achieved by adding to the slurry a hydrolyzing agent, such as an acid. Any suitable hydrolyzing agent, or combination of agents, capable of lysing the cells and breaking down complex carbohydrates and lipids may be used. Exemplary hydrolyzing acids may include strong acids, mineral acids, or organic acids, such as sulfuric, hydrochloric, phosphoric, or nitric acid. These acids are all capable of accomplishing the goals stated above. When using an acid, the pH of the slurry should be less than 7, such as from about 1-6, about 1.5-4, or about 2-2.5.

In addition to strong acids this digestion may also be accomplished using enzymes alone or in combination with acids that can break down plant material. However, any such enzymes or enzyme/acid combinations would also be capable of breaking down the complex polysaccharides to their respective oligosaccharides or monosaccharides as well as complex lipids to free fatty acids.

In some embodiments, the acid or enzymes, or a combination thereof, may be mixed with water to form a hydrolyzing solution. However, in other embodiments, the hydrolyzing agent may be directly added to the slurry.

(2) Base Hydrolysis

After the initial acidic hydrolysis, a secondary base hydrolysis may be performed to digest and break down any remaining whole algae cells; hydrolyze any remaining complex polysaccharides and lipids and bring those polysaccharides and lipids into solution; convert all free fatty acids to their salt form, or soaps; and to break chlorophyll molecules apart.

In this secondary hydrolysis, the biomass in the slurry is mixed with a basic hydrolyzing agent to yield a pH of greater than 7, such as about 8-14, about 11-13, or about 12-12.5. Any suitable base may be used to increase in pH, for example, sodium hydroxide, or other strong base, such as potassium hydroxide may be used. Temperature, time, and pH may be varied to achieve more efficient digestion.

This basic slurry may be optionally heated. When heated, the slurry may reach temperatures of from about 1-200° C., such as about 20-100° C., about 50-95° C., or about 90° C. When temperatures above 100° C., or the boiling point of the solution are used, an apparatus capable of withstanding pressures above atmospheric pressure may be employed. In some embodiments, depending on the type of algae, the type and concentration of acid used for hydrolysis, the outside temperature conditions, the permissible reaction time, and the conditions of the slurry, heating may be omitted. Heating may occur prior to, during, or after addition of a hydrolyzing agent.

In addition, the basic slurry may be optionally mixed either continuously or intermittently. Alternatively, a hydrolysis reaction vessel may be configured to mix the slurry by convection as the mixture is heated.

Basic hydrolysis may be permitted to take place for a suitable period of time depending on the temperature of the slurry and the concentration of the hydrolyzing agent. For example, the reaction may take place for up to 72 hours, such as from about 12-24 hours. If the slurry is heated, then hydrolysis may occur at a faster rate, such as from about 15-120 minutes, 30-90 minutes, or about 30 minutes.

During this basic hydrolysis, chlorophyll is hydrolyzed to the porphyrin head and phytol side chain, as well as complex polysaccharides are hydrolyzed to oligosaccharides or their respective monosaccharide component.

B. Biomass and Aqueous Phase Separation

Under the condition of elevated pH, the biomass may be separated from the aqueous solution. This separation is performed while the pH remains high to keep the lipids in their soap form so that they are more soluble in water, thereby remaining in the water, or liquid, phase. Once the separation is complete, the liquid phase is kept separate and the remaining biomass may be optionally washed with water to help remove any residual soap molecules. This wash water may also be collected along with the original liquid phase. Once the biomass is washed it may be taken to the next phase of the process. The liquid phase may be processed further to derive other useful products, such as biodiesel as described in U.S. application Ser. No. 13/660,161, filed on Oct. 25, 2012.

The resulting biomass, containing sugars, may then be taken through the exemplary ABE production process described below, or some other suitable ABE production method.

III. ABE Production

A. Bacterial Producers

Any suitable bacteria or microorganism capable of metabolizing algal biomass into solvents may be used. At least one Clostridium species or group of species may be used to ferment the algal biomass into ABE. For example, suitable Clostridium species may include, Clostridium saccharoperbutylacetonium, Clostridium acetobutylicum, Clostridium beijerinckii, or any suitable Clostridium bacteria isolated from the environment.

B. Fermentation

ABE fermentation is typically characterized by two distinct phases of metabolism, acidogenesis and solventogenesis. Acidogenesis occurs during log phase of growth, whereas solventogenesis occurs late log phase to early stationary phase of growth. The primary acids produced during acidogenesis are acetic and butyric acid. Clostridia re-assimilate the acids produced during acidogenesis and produce acetone, butanol, and ethanol as metabolic byproducts. The pH-acid effect from acidogenesis plays a key role in the onset of solventogenesis. See, Li et al., Performance of batch, fed-batch, and continuous A-B-E fermentation with pH-control, 102 Bioresource Technology.4241-4250 (2011).

Any suitable culture medium may be used. Culture medium is used to support the growth of microorganisms, and can be modified to support microbial growth or derive production of certain bio-products. Medium recipes contain vitamins, minerals, buffering agents, nitrogen sources, and carbon sources necessary for bacterial growth. The carbohydrates within algal cells are the carbon source used to drive ABE production throughout the claims. For example, the following culture medium, referred to as T-6, may be used.

T-6 Medium (Approximate formula per liter)

| Component | Amount | |
|---|---|---|
| Tryptone | 6.0 | g |
| Yeast extract | 2.0 | g |
| KH2PO4 | 0.5 | g |
| MgSO4•7H20 | 0.3 | g |
| FeSO4 7H20 | 10 | mg |
| Ammonium acetate (38.9 mM) | 3.0 | g |
| Cysteine hydrochloride | 0.5 | g |
| Glucose or Algae or other substrate | 5.0-15.0% | (w/v) |

Adjust pH to 6.5 with NaOH

The medium may be formulated to contain about 1 to about 20% processed algae by weight per liter of medium, such as about 4 to about 15%, 5 to about 8%, or 6%.

The other components of the T-6 medium may be varied and adjusted based upon desired growth parameters and/or culturing conditions. In addition, other suitable mediums may include RCM media and TYA media, both of which have been shown to provide suitable nutrients for ABE fermentation with algae as substrate.

The medium may be supplemented with enzymes and/or sugars to help initate primary growth. Suitable enzymes include cellulases and xylanases in amounts ranging from about 10 to about 250 units of enzyme. Suitable sugars include glucose, starch, arabinose, galactose, and xylose in amounts ranging from about 0.1% to about 1.0%. In some embodiments, cheese whey or whey permeate may be used to substitute or augment media components. These nutrient sources have advantages because they are a waste stream of milk product processing and, as such, may have cost advantages over other nutrient sources.

Once T-6 media constituents are mixed to homogeneity, the media may be neutralized to a pH of about 7, such as about 6.5. The medium may then be modified by any suitable technique to create an anaerobic environment. Suitable techniques for creating such an environment include bubbling the medium with O₂-free N₂ gas for a suitable period of time.

Prior to or after the creation of the anaerobic environment, the medium may be optionally sterilized.

The medium may be inoculated with at least one Clostridium species. The concentration of bacteria may be varied, depending on the culture vessel and scale of the fermentation. Prior to or after inoculation, the bacterium may be heat shocked to a temperature of about 70° C. for a suitable period of time to germinate the spores. The bacterium may also be incubated in a growth medium at optimal temperature prior to inoculation to allow the spores to become vegetative prior to transferring to the growth medium. After inoculation, the fermentation vessel head space, if any, may be flushed with N₂ gas to ensure optimal anaerobic growth conditions.

The culture may be incubated at about 35° C. throughout. Typically, 48 hours is needed for T-6 glucose cultures containing spores of Clostridium saccharoperbutylacetonium to reach mid-log phase, though fermentation times may vary depending on the vessel size, inoculation concentration, and temperature. T-6 algae media fermentations may be conducted for about 96 hours to reach optimal ABE production. T-6 glucose fermentations may be used as the positive control, whereas T-6 media without a carbon source may be used as the negative control throughout.

C. ABE Purification

Any suitable purification method may be employed. In some embodiments, distillation may be used for purifying the various fermentation products. Distillation is used widely for alcoholic beverages, as well as for other types of fermented solutions, particularly acetone, butanol, and ethanol. When distillation is employed, purification is accomplished based on different boiling points from one compound to another. By heating a mixture to a temperature just above each solvents boiling point, the desired compound evaporates and then condenses independently to acquire purified solvents.

In some embodiments, each of the fermentation products may be purified; however, in other embodiments, only a select product or group of products may be purified. In particular, because the yield for acetone and butanol are higher than that of ethanol, some purification processes only purify acetone and butanol, while other fermentation products are flared off or otherwise discarded.

Other suitable purification methods may be employed, such as absorption, membrane pertraction, extraction, and gas stripping. See, e.g., Kaminski et al., Biobutanol - Production and Purification Methods, Ecological Chemistry and Engineering S., Vol. 18, No: 1 (2011).

The following examples are illustrative only and are not intended to limit the disclosure in any way.

### EXAMPLES

### Example 1

### Biomass Processing

To a glass test tube 100 mg of lyophilized algal biomass was added. One mL of a 1 Molar Sulfuric acid solution is added to the test tube and the test tube was then sealed using a PTFE lined screw cap and gently mixed to create a homogenous slurry. This slurry was then placed in a Hach DRB-200 heat block pre-heated to 90° C. This slurry is allowed to digest for 30 minutes with mixing at the 15 minute mark.

Once the first 30 minute digestion period was completed, the test tube was removed from the heat source and 0.75 mL of a 5 Molar Sodium Hydroxide solution was added to the test tube. The test tube was immediately resealed and returned to the heat source for 30 minutes. Mixing at 15 minutes was again provided.

Once the base hydrolysis above was completed, the test tube was removed from the heat source and allowed to cool in a cold water bath. Once cooled the test slurry was centrifuged using a Fisher Scientific Centrific Model 228 centrifuge. The upper aqueous phase was removed and collected in a separate test tube. To the remaining biomass 1 mL of deionized water as added and vigorously mixed. The slurry was re-centrifuged, and the liquid phase collected and added to the previously collected liquid phase. The liquid phase was then removed from the process and processed biomass was taken for further processing.

### Example 2

### ABE Production using Processed Biomass and No Supplementation of Enzymes or Sugar

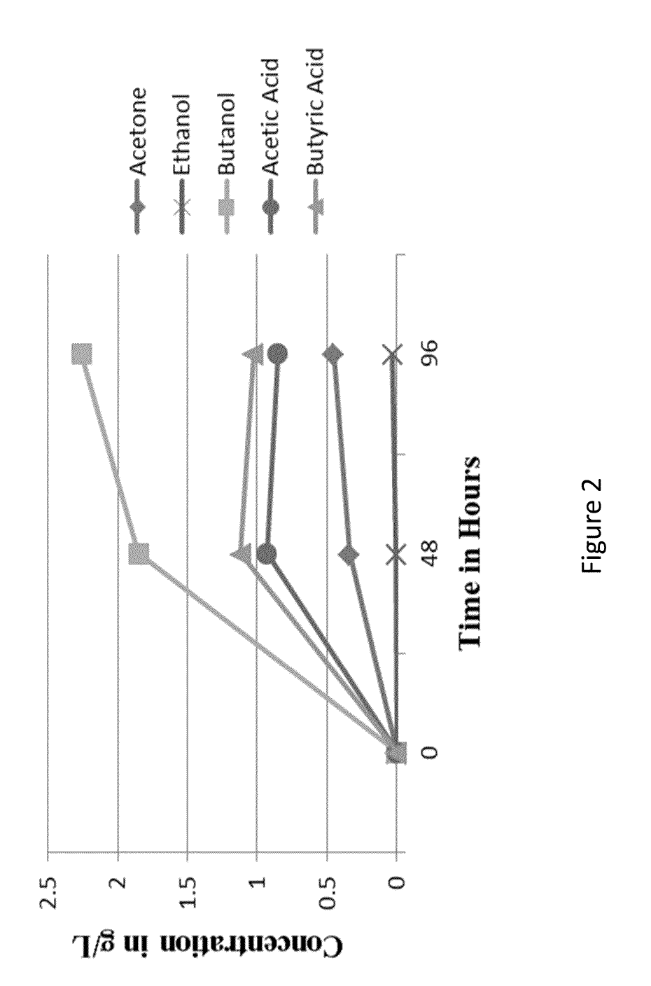

10% algal biomass was processed according the parameters described in Example 1. The T-6 media constituents were mixed to homogeneity, and the media neutralized to pH 6.5, and the media was then dispensed into serum vials. These vials were then bubbled with O₂ free N₂ gas for 10 minutes to remove any O₂ (thus generating an anaerobic environment). Once this was performed, the vials were sealed, crimped, and sterilized. After sterilization, 1 ml of a concentrated spore suspension containing Clostridium saccharoperbutylacetonium was transferred to T-6 glucose media anaerobically. After inoculation, the growth media containing spores was heat shocked at 70° C. for 10 minutes to germinate spores and incubated at optimal temperature. This step allowed the spores to become vegetative prior to transferring into T-6 algae media. After the T-6 glucose culture reached mid-log phase, a 10% inoculum of mid-log phase cells was transferred into T-6 algae media (containing 10% processed algae) anaerobically. After fermentation media was inoculated, the headspace was flushed with O₂ free N₂ gas for 5 minutes to ensure optimal growth conditions and O₂ removal. The culture was then incubated at 35° C. throughout for 48 hours to reach mid-log phase. The fermentation was conducted for 96 hours to reach optimal ABE production. The mean yield results of two replicates of are illustrated in FIG. 2.

### Example 3

### ABE Production using Processed Biomass and Enzymes

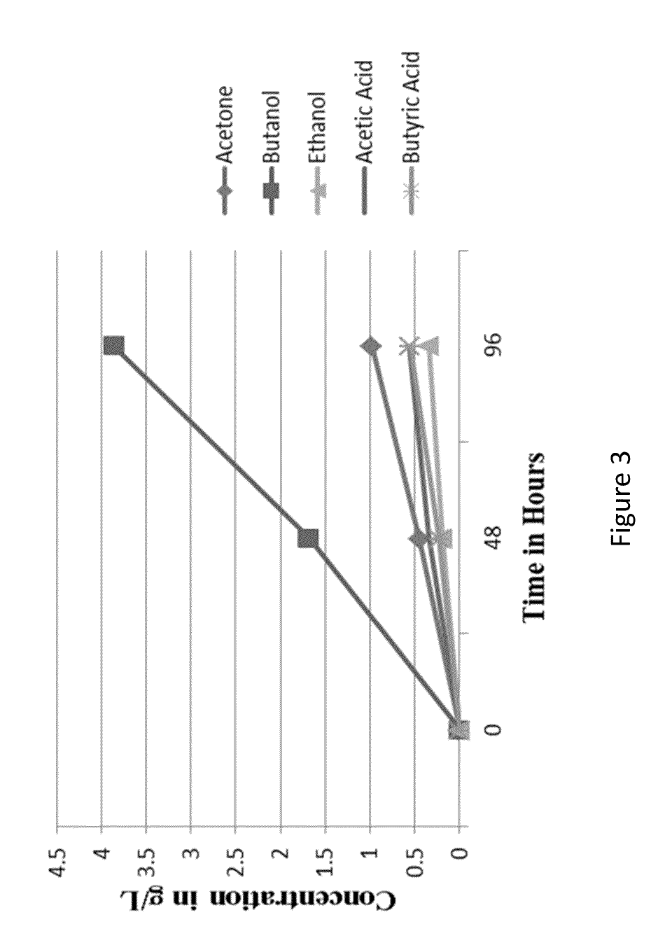

10% algal biomass was processed according the parameters described in Example 1. The process biomass was fermented as described in Example 2 with the supplementation of 250 units of xylanase and 100 units of cellulose added to the fermentation. The yield results are illustrated in FIG. 3.

### Example 4

### ABE Production using Processed Biomass and Sugar

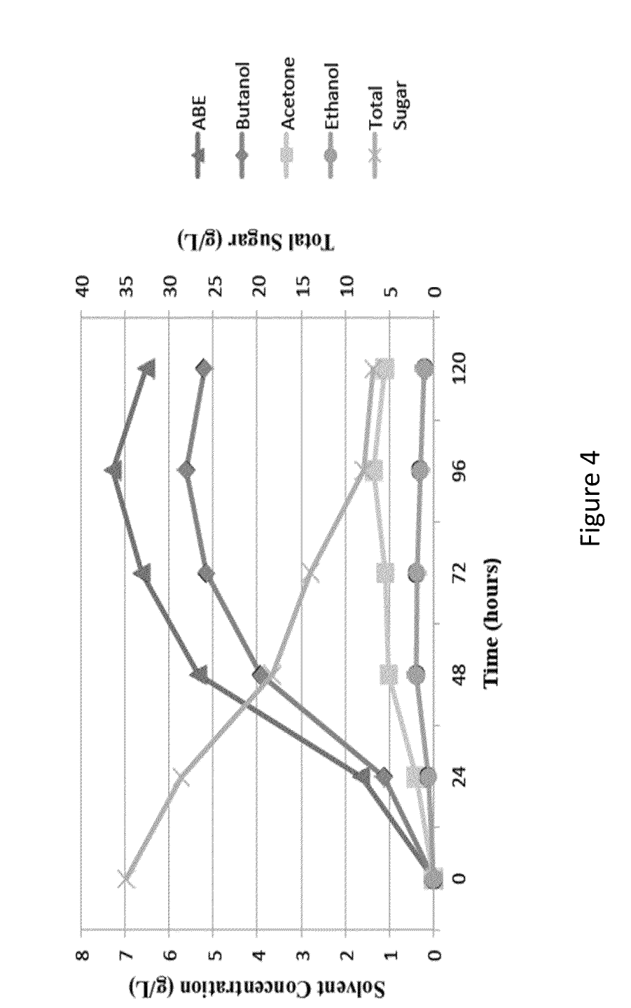

The same process as described in Example 2 was repeated, this time supplementing only with 1% dextrose. The yield results are illustrated in FIG. 4.

### Example 5

### ABE Production using Pretreated Algae and Enzymes

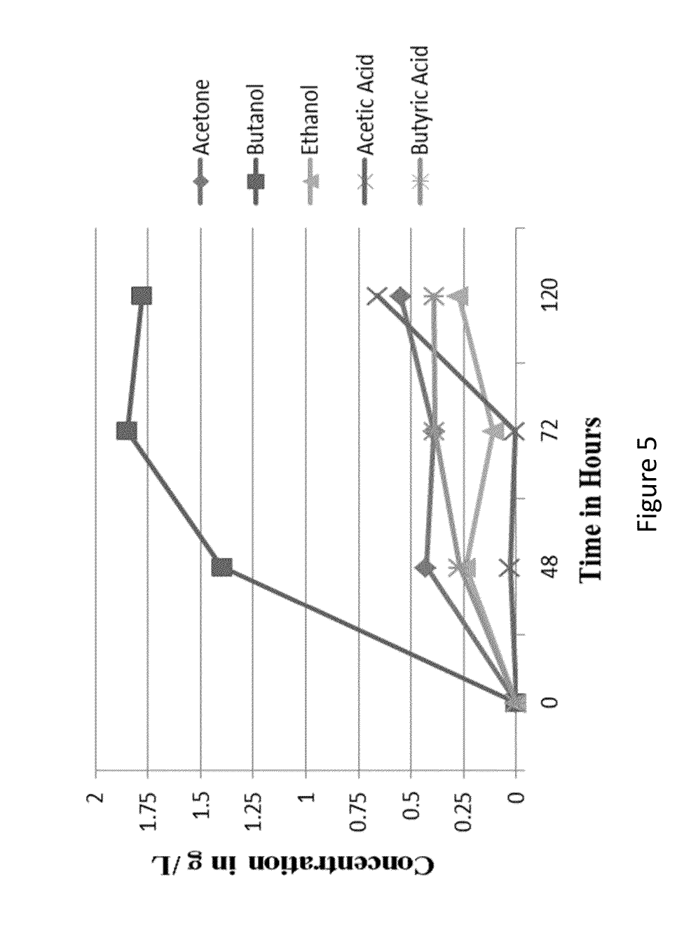

Dried algae was crushed using a blender and then pretreated with 250 mM sulfuric acid for 30 min at 120° C. Acid and solvent production from Clostridium saccharoperbutylacetonium using 10% algae supplemented with xylanase and cellulase enzymes as described in Example 2 was undertaken. The yield results are illustrated in FIG. 5.

### Example 6

### ABE Production using Pretreated Algae and Enzymes

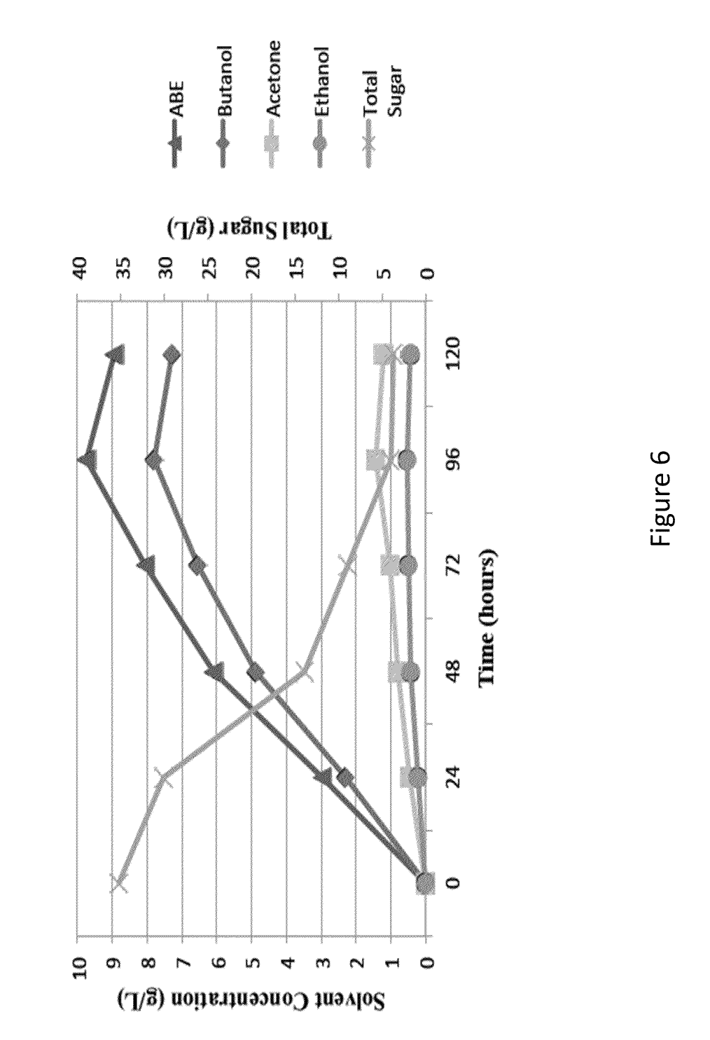

Dried algae was crushed using a mortar and pestle and then pretreated with 250 mM sulfuric acid for 30 min at 120° C. Acid and solvent production from Clostridium saccharoperbutylacetonium using 10% algae supplemented with xylanase and cellulase enzymes as described in Example 2 was undertaken. The yield results are illustrated in FIG. 6.

### Example 7

### ABE Production using Non-Pretreated Whole Cell Algae

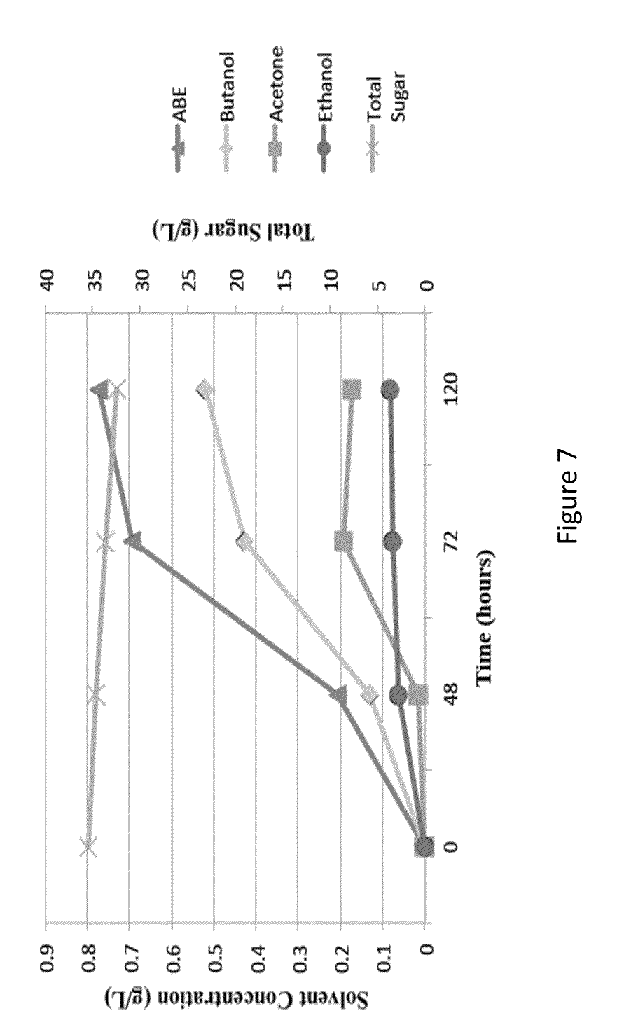

Dried algae was used in T-6 media without any chemical or mechanical modifications to the algae cells. The algae was fermented according to the fermentation conditions outlined in Example 2, except that dried, unprocessed algae was used and a 5% inoculum was used for a 24 hour culture in RCM media. The yield results are illustrated in FIG. 7.

### Example 8

### Gas Chromatography (GC)

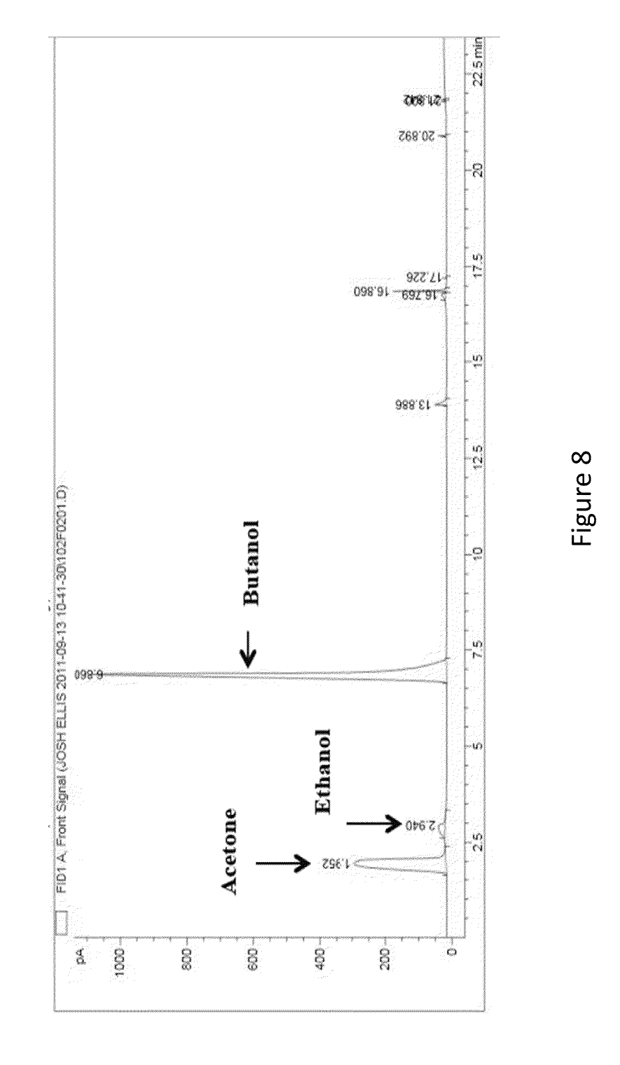

A GC chromatogram, used to measure or quantify ABE, using clarified culture supernatant the method described in Example 4 is shown in FIG. 8. The protocol for measuring ABE via GC analysis is as follows:
Instrument: Agilent Technologies 7890A GC system.
Column specs: Restek Stabiwax-DA, 30 m, 0.32 mmlD, 0.25 um df column.
Inlet: initial 30 C for 1 min; ramp 5 C/min up to 100 C; ramp 10 C/min up to 250 C.
Column: flow 4 ml/min; pressure 15.024 psi, Avg velocity 53.893 cm/sec; holdup time 0.92777 min.
Oven: initial 30 C for 1 min; ramp 5 C/min up to 100 C (no hold time); ramp 20 C/min up to 225 C (no hold time); ramp 120 C/min up to 250 C and hold for 2 min.
FID: Heater at 250 C; H2 flow at 30 ml/min; Air flow at 400 ml/min; makeup flow (He) at 25 ml/min.
Miscellaneous: 1 μl injection volume, and Helium as carrier gas.

### Example 9

### ABE Production using Whey

Cheese whey or whey permeate may be used to substitute media components. Acid and solvent production from Clostridium saccharoperbutylacetonium using 6% whey, instead of 10% algae, was undertaken under the culture conditions described in Example 2. The final ABE yields were as follows: approximately 15 g/I butanol, 4 g/I acetone, and 1 g/I of ethanol.

### Example 10

### ABE Production using Whey and Pretreated Algae

ABE production using whey as media constituents as oppose to T6 media components, or any other viable media, with algae as additional substrate are additional methods for producing ABE. Supplementing algae using similar methods as in Example 2, Example 3, Example 4, Example 5, and Example 6 with whey permeate or cheese whey is a viable option to producing ABE.

It will be appreciated that various of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art, and are also intended to be encompassed by the following claims.

What is claimed is:

1. A method of producing solvents from algae, the method comprising:
processing algae to yield processed biomass,
fermenting the processed biomass with a Clostridium bacteria to yield solvents.

2. The method of claim 1, wherein processing algae comprises:
hydrolyzing a slurry comprising algae and water by adding an acidic hydrolyzing agent to yield an acidic slurry,
hydrolyzing the acidic slurry by adding a basic hydrolyzing agent to yield a basic slurry, and
separating biomass from an aqueous phase to yield processed biomass.

3. The method of claim 2, wherein the slurry has a solid content of about 4-25%.

4. The method of claim 2, wherein the acidic hydrolyzing agent is selected from the group consisting of a strong acid, a mineral acid, sulfuric acid, hydrochloric acid, phosphoric acid, and nitric acid.

5. The method of claim 2, wherein the acidic slurry has a pH of from about 1.5-4.

6. The method of claim 2, wherein the acidic slurry is heated to a temperature of from about 50-120 ° C.

7. The method of claim 2, wherein the basic hydrolyzing agent is selected from the group consisting of a strong base, sodium hydroxide, and potassium hydroxide.

8. The method of claim 2, wherein the basic slurry has a pH of from about 8-14.

9. The method of claim 2, wherein the basic slurry is heated to a temperature of from about 50-120 ° C.

10. The method of claim 1, wherein processing algae comprises mechanically shearing the algae.

11. The method of claim 1, wherein the Clostridium bacteria are selected from the group consisting of Clostridium saccharoperbutylacetonium, Clostridium acetobutylicum, and Clostridium beijerinckii.

12. The method of claim 1, wherein the solvents are selected from the group consisting of butanol, acetone, and ethanol.

13. The method of claim 1, further comprising purifying the solvents.

14. The method of claim 13, wherein purifying comprises distillation.

15. The method of claim 1, wherein fermenting the processed biomass comprises suplementing the biomass with a suplement selected from the group consisitng of sugar, dextrose, lactose, whey, whey permeate, and chesse whey.

16. The method of claim 1, wherein fermenting the processed biomass comprises suplementing the biomass with an enzyme selected from the group consisitng of xylanase and cellulase.

17. A method of of producing solvents from algae, the method comprising fermenting the algae with a Clostridium bacteria to yield solvents.

18. The method of claim 17, wherein the solvents comprise at least one of acetone, butanol, and ethanol.

19. A method of of producing solvents from whey, the method comprising fermenting the whey with a Clostridium bacteria to yield solvents.

20. The method of claim 19, wherein the solvents comprise at least one of acetone, butanol, and ethanol.
